(19) United States
(12) Patent Application Publication
(10) Pub. No.: US 2025/0099564 A1
(43) Pub. Date: Mar. 27, 2025

(54) A Novel Poultry Salmonella Vaccine and Diagnostic Methodology to Control Foodborne Salmonellosis

(71) Applicants: University of Florida Research Foundation, Incorporated, Gainesville, FL (US); THE PENN STATE RESEARCH FOUNDATION, UNIVERSITY PARK, PA (US)

(72) Inventors: Subhashinie KARIYAWASAM, Gainesville, FL (US); Dona Saumya Sewwandi WIJETUNGE, UNIVERSITY PARK, PA (US)

(21) Appl. No.: 18/700,490

(22) PCT Filed: Oct. 11, 2022
(86) PCT No.: PCT/US2022/077887

§ 371 Date: Apr. 11, 2024

Related U.S. Application Data
(60) Provisional application No. 63/254,482, filed on Oct. 11, 2021.

(51) Int. Cl. A61K 39/112; A61K 39/00; A61K 39/108; A61P 31/14; C12N 15/70; C12Q 1/689

(52) U.S. Cl. CPC A61K 39/0275; A61K 39/0258; A61P 31/14; C12N 15/70; C12Q 1/689; A61K 2039/522; A61K 2039/523; A61K 2039/545; A61K 2039/552

(57) ABSTRACT

A vaccine for treating Salmonella enteriditis that includes an immunogenically effective amount of a Salmonella enteritidis protein InvG, and optionally a pharmaceutically acceptable carrier is described. Methods of using compositions that include the Salmonella Enteritidis protein InvG or a delivery vector that expresses Salmonella Enteritidis protein InvG for immunizing poultry against Salmonella Enteritidis are also described.

### GOVERNMENT SUPPORT

This invention was made with government support under 2020-67017-33079 awarded by The United States Department of Agriculture, National Institutes of Food & Agriculture. The government has certain rights in the invention.

### BACKGROUND

Salmonella enterica subsp. enterica (Salmonella) is the leading cause of bacterial foodborne disease in the United States, with approximately 1.2 million human cases annually (50). This includes approximately 20,000 hospitalizations, 450 deaths, and an estimated cost of 2.6 billion dollars in healthcare costs (15, 50). The actual numbers can be much higher than that, due to under-reporting, and it has been estimated that 29.3 cases of salmonellosis occur for every case that is laboratory confirmed and reported (15). Although invasive Salmonella infections are rare, they can progress into severe and life-threatening infections (15). In recent years, increasing resistance of nontyphoidal Salmonella to antibiotics, most importantly two clinically important drugs, ceftriaxone and ciprofloxacin, has further escalated the public health threats posed by these bacteria (14, 20). There was also a significant increase in nontyphoidal Salmonella resistance to more than three antibiotic classes, i.e. multidrug resistance, according to the latest report from Healthy People 2020 (14, 46).

Salmonella can be isolated from a multitude of food products (12). Annual culture-confirmed human infection surveys regarding Salmonella indicate that the five most prevalent serovars in decreasing order are Enteritidis, Newport, Typhimurium, and Javiana, and Heidelberg in the United States (13, 26). Except S. Javiana, which is not often associated with products regulated by the agency, all other serotypes were most commonly associated with shell eggs, poultry meat, and poultry products (26, 49). The intestines of chickens and other poultry species are asymptomatically colonized by nontyphoidal Salmonella, as a result of horizontal or vertical transmission of bacteria (4). In a single flock, as many as 65% of the birds may carry Salmonella. Salmonella serovars colonizing the poultry intestinal tract depend on the geographic location and the time of the year, and a single bird may be colonized with more than one serovar at any given time (8, 45). As such, the poultry industry is under increasing consumer and regulatory pressure to guarantee food safety and meet export requirements.

In attempts to reduce Salmonella-related foodborne outbreaks, many farm-to-folk type regulatory programs have been enforced by the United States Department of Agriculture National Poultry Improvement Plan (USDA-NPIP), Food and Drug Administration (FDA), and United States Department of Agriculture Food Safety and Inspection Service (USDA-FSIS) (23, 64, 66). These programs are directed at improving biosecurity measures, monitoring and sampling, vaccination, and poultry carcass treatment (62, 64, 66). Despite these regulatory measures, foodborne illnesses due to nontyphoidal Salmonella continue to be a major food safety and public health issue. In fact, the incidence rates remain much higher than the objective of Healthy People 2020, which strives to reduce nontyphoidal Salmonella infections to 11. (4) per 100,000 people, and to prevent an increase in the proportion of antibiotic resistant nontyphoidal Salmonella from humans (14).

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1A-B are genetic maps showing a comparison of S. Enteritidis SEE1 genome with the other sequenced strains of S. Enteritidis. FIG. 1A. Genome content, FIG. 1B. whole-genome Single Nucleotide Polymorphism (SNP) comparison.

FIG. 2A-D are bar graphs showing bacterial burden in the intestinal tract and fecal shedding. Bacterial counts of the small intestine (FIG. 2A), cecum (FIG. 2B), colon (FIG. 2C), as well as fecal shedding differences (FIG. 2D), were taken from three experiments, normalized with corresponding controls. * indicates a statistical significance.

FIG. 3A-B are bar graphs showing the overall size (FIG. 3A), weight (FIG. 3B) biometrics of the ceca.

FIG. 3 C1-3 are pictures showing pathology-driven morphological biometrics of the ceca changes were determined for the mice infected with SEE1 from three different sources. SEE1 grown in egg yolk appears to cause greater pathological changes in the ceca, as compared to SEE1 grown in LB broth (FIG. 3C1) or passed through mice (FIG. 3C3).

FIG. 4A-D are slides showing changes in the histopathology of ceca infected with SEE1 from various sources. Healthy ceca from an uninfected mouse (FIG. 4A), cecal sections of mice infected with SEE1 grown in egg yolk (FIG. 4B), SEE1 passed through mice (FIG. 4C), and SEE1 grown in LB broth (FIG. 4D). GC (Goblet Cells), EP (Epithelial Cells), LP (Lamina Propria), TM (Tunica Muscularis), MM (Muscularis Mucosa), PMN (Polymorphonuclear leukocytes), SME (Sub-mucosal Edema), UL (Ulceration)

FIG. 5 is a bar graph of cytokine analysis of ceca of mice infected with SEE1 from three sources. Proinflammatory and anti-inflammatory cytokine profiles had been determined by ELISA for ceca of mice infected with SEE1 from egg yolk, LB broth (four mice), and passed through mice.

FIG. 6 is an SDS-page gel showing outer membrane profiles of SEE1 grown in egg yolk and LB broth.

FIG. 7A-B are SDS PAGE gels confirm of ˜60 KDa protein as InvG.

FIG. 8 is a bar graph showing adherence deficiency of the mutant strain. Results are expressed as the relative bacterial numbers, compared with the WT bacteria grown in egg yolk as 100%.

FIG. 9 is an SDS PAGE gel showing two bacterial clones expressing the InvG protein. The red arrow indicates the ˜62 KDa InvG protein.

FIG. 10 is the genomic map of PSUO78 (GenBank accession CP012112.1), an egg isolate of APEC, in comparison to other sequenced APEC genomes.

FIG. 11 shows a summary of a high throughput rapid test for S. Enteritidis detection in egg yolk. FIG. 11A: After forming the base circuit pattern with interdigitated electrodes, an Nd:Yag laser is used to perforate the carbon circuit (10 μm holes). FIG. 11B shows the use of a emulsion-coated cellulose paper or nitrocellulose membranes in a flow system.

FIG. 12 shows an experimental design for assessing the immunogenicity and efficacy of InvG against a challenge of S. Enteritidis.

FIG. 13 shows an experimental design for assessing the efficacy of passively transferred anti-InvG antibodies against S. Enteritidis challenge.

FIG. 14 shows an experimental design for assessing the efficacy of passively transferred anti-InvG antibodies against a heterologous challenge of Salmonella.

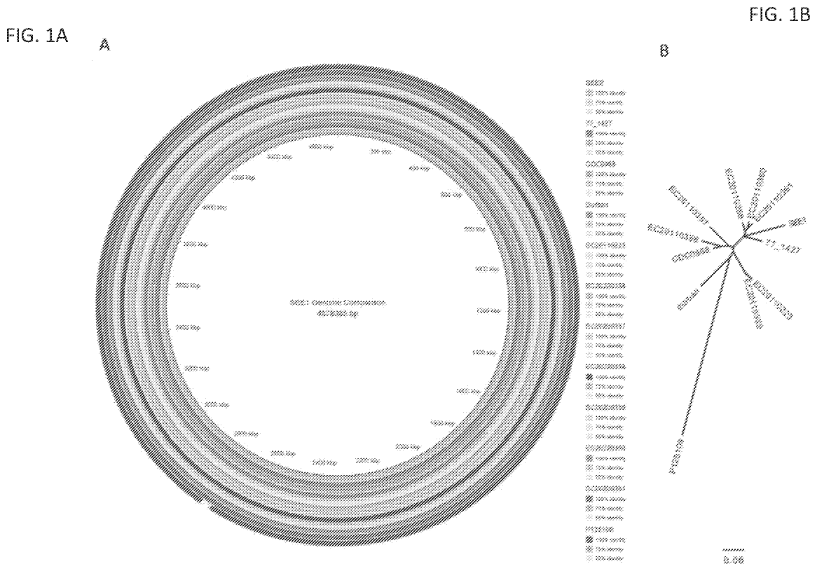

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “adjuvant” as used here refers to an agent that induces in an inoculated animal host a heightened means to withstand infection and to elicit an improved level of acquired immunity in the host when exposed to an antigen, vaccine or pathogen or part thereof. Adjuvants can also enhance display of natural barriers to infection by pathogens and diminish the ability of pathogens to infect, colonize or cause disease.

The term “administering in ovo” or “in ovo administration,” as used herein, unless otherwise indicated, means administering an adjuvant composition to a bird egg containing a live, developing embryo by any means of penetrating the shell of the egg and introducing the adjuvant composition. Such means of administration include, but are not limited to, in ovo injection of the adjuvant composition.

The term “avirulent mutant” as used herein, unless otherwise indicated, refers to an E. coli that possesses one or more mutations that eliminate or diminish means of decreasing induction of effective immunogenicity in a poultry subject. In one example, the E. coli possesses an aroA and/or asd deletion (e.g.). In a specific example, the avirulent mutant E. coli strain is APEC PSUO78 comprising both an ΔaroA and/or Δasd deletion or disruption.

The terms “poultry” and “poultry subjects” as used herein, are intended to include males and females of any avian or bird species, and in particular are intended to encompass poultry which are commercially raised for eggs, meat or as pets. Accordingly, the terms “poultry” and “poultry subject” encompass chickens, turkeys, ducks, geese, quail, pheasant, parakeets, parrots, cockatoos, cockatiels, ostriches, emus and the like. Commercial poultry includes broilers and layers, which are raised for meat and egg production, respectively.

The terms “capture probe” as used herein, unless otherwise indicated, refers to antibodies, aptamers, lectins, and oligopeptides that target extracellular membrane proteins to allow for detection of pathogens.

The terms “clone”, “cloning”, or “cloned” as used herein, unless otherwise indicated, refers to the process of making multiple molecules. Molecular cloning is a set of experimental methods known in the art of molecular biology that are used to assemble recombinant DNA and protein molecules and to direct their replication or expression within host organisms.

The term “challenge” as used herein, unless otherwise indicated, means to expose the immune system to pathogenic organisms or antigens to evoke an immunologic response. A challenge can include multiple organisms (heterologous) or individual organisms (homologous). An example of a challenge includes, but is not limited to, testing the efficacy of a vaccine.

The term “effective amount” as used herein refers to an amount effective to achieve an intended purpose.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the invention. Preferably, the host will display either a protective immunological response or a therapeutically response.

The term “immunogenic” or “immunogenic activity” refers to the ability of a polypeptide to elicit an immunological response in a subject (e.g., a mammal). An immunological response to a polypeptide is the development in an animal of a cellular and/or antibody-mediated immune response to the polypeptide. Usually, an immunological response includes but is not limited to one or more of the following effects: the production of antibodies, B cells, helper T cells, suppressor T cells and/or cytotoxic T cells, directed to an epitope or epitopes of the polypeptide.

The term “immunogenic composition” refers to a composition that comprises at least one antigen, which elicits an immunological response in the host to which the immunogenic composition is administered. Such immunological response may be a cellular and/or antibody-mediated immune response to the immunogenic composition of the invention. Preferably, the immunogenic composition induces an immune response and, more preferably, confers protective immunity against one or more of the clinical signs of a Salmonella infection. The host is also described as “poultry subject”.

The term “immunogenically effective amount,” as used herein, unless otherwise indicated, means an amount or dose of a composition sufficient to induce an innate immune response in the treated birds that is greater than the inherent immunity of non-inoculated birds. An immunogenically effective amount in any particular context can be routinely determined using methods known in the art.

The term “pathogen” as used herein refers to a bacteria, virus, fungus or parasite that is capable of infecting and/or causing adverse symptoms in a subject. Examples of specific pathogens include, but are not limited to, Salmonella spp, Escherichia coli strains, Clostridium spp, Campylobacter spp and to influenza virus (e.g. avian influenza virus).

As used herein, the terms “peptide,” “polypeptide,” or “protein” are used interchangeably herein and are intended to encompass a singular “polypeptide” as well as plural “polypeptides,” and refers to a molecule composed of monomers (amino acids) linearly linked by amide bonds (also known as peptide bonds). The terms “peptide” and “polypeptide” refer to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “peptide” and “polypeptide”. The terms apply to amino acid polymers in which one or more amino acid residues is an artificial chemical analogue of a corresponding naturally occurring amino acid, as well as to naturally occurring amino acid polymers. Non-limiting examples of artificial amino acid residues include norleucine and selenomethionine. An amino acid residue is a molecule having a carboxyl group, an amino group, and a side chain and having the generic formula EbNCHRCOOH, where R is an organic substituent, forming the side chain. An amino acid residue, whether it is artificial or naturally occurring, is capable of forming a peptide bond with a naturally occurring amino acid residue.

The immunogenic polypeptides used in the presently disclosed compositions and methods can be recombinantly produced, chemically synthesized, or purified from a biological sample. In some embodiments, the immunogenic polypeptide is an isolated polypeptide

The term “Salmonella enterica protein InvG” as used herein refers to an amino acid sequence comprising SEQ ID NO: 1. Unless specified otherwise, Salmonella enterica protein InvG includes fragments or variants of SEQ ID NO: 1 that induce an immune response.

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art. The determination of percent identity between two sequences can be accomplished using a mathematical algorithm. For example, percent identity of an amino acid sequence can be determined using the Smith-Waterman homology search algorithm using an affine 6 gap search with a gap open penalty of 12 and a gap extension penalty of 2, BLOSUM matrix 62. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic Version G. The Smith-Waterman homology search algorithm is taught in Smith and Waterman (1981) Adv. Appl. Math 2:482-489, herein incorporated by reference. Alternatively, the alignment program GCG Gap (Wisconsin Genetic

Computing Group, Suite Version 10.1) using the default parameters may be used. The GCG Gap program applies the Needleman and Wunch algorithm and for the alignment of nucleotide sequences with an open gap penalty of 3 and an extend gap penalty of 1 may be used. Another preferred, non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 2/5:403. BLAST nucleotide searches can be performed with the NBLAST program, score=100, wordlength 12, to obtain nucleotide sequences having sufficient sequence identity. BLAST protein searches can be performed with the XBLAST program, score=50, wordlength=3, to obtain amino acid sequences having sufficient sequence identity. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See www.ncbi.nlm.nih.gov. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

By “variant” is intended substantially similar sequences. Thus, immunogenic variants include sequences that are functionally equivalent to the protein sequence of interest and retain immunogenic activity. Generally, amino acid sequence variants of the invention will have at least 40%, at least about 50%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to a respective amino acid sequence. In a specific embodiment, an immunogenic variant comprises at least 95% sequence identity with SEQ ID NO: 2.

### DETAILED DESCRIPTION

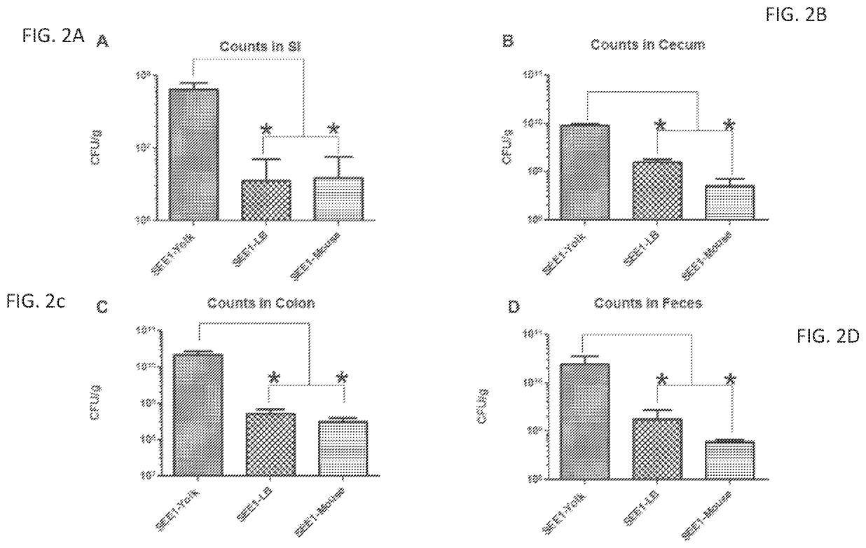

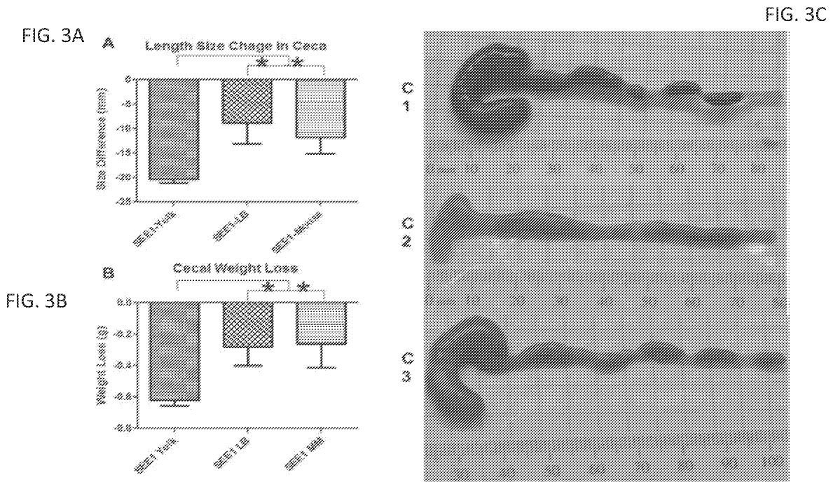

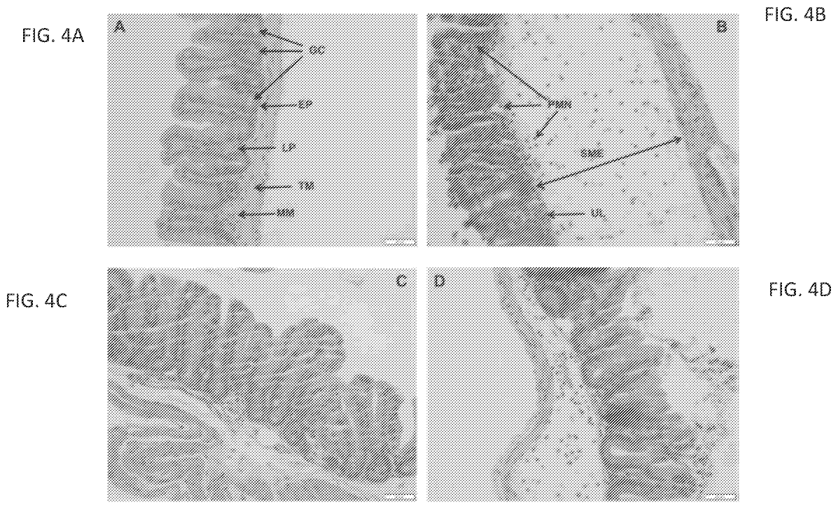

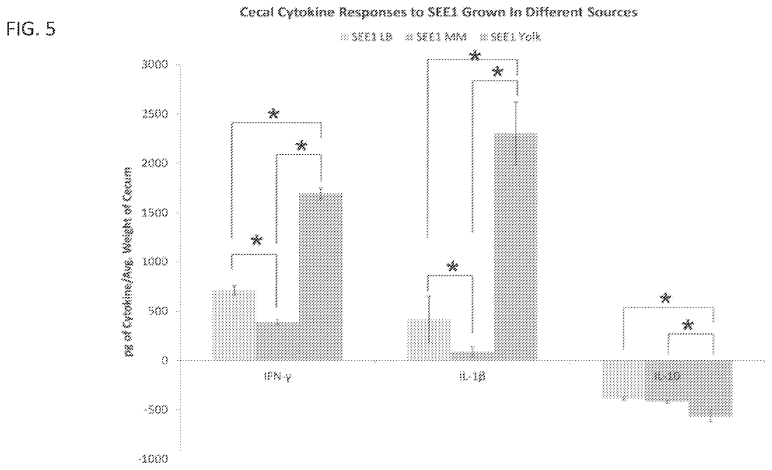

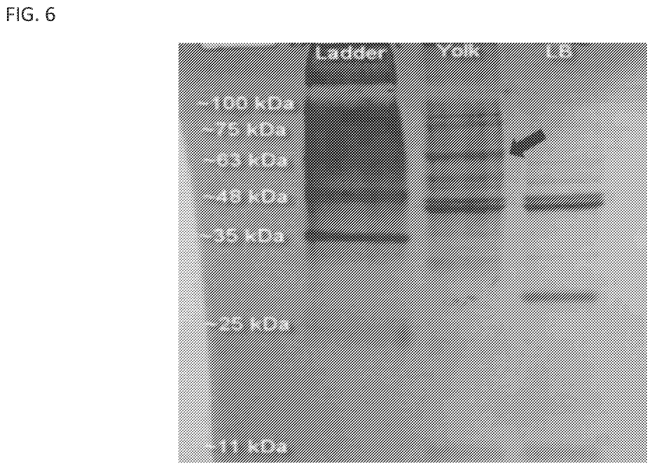

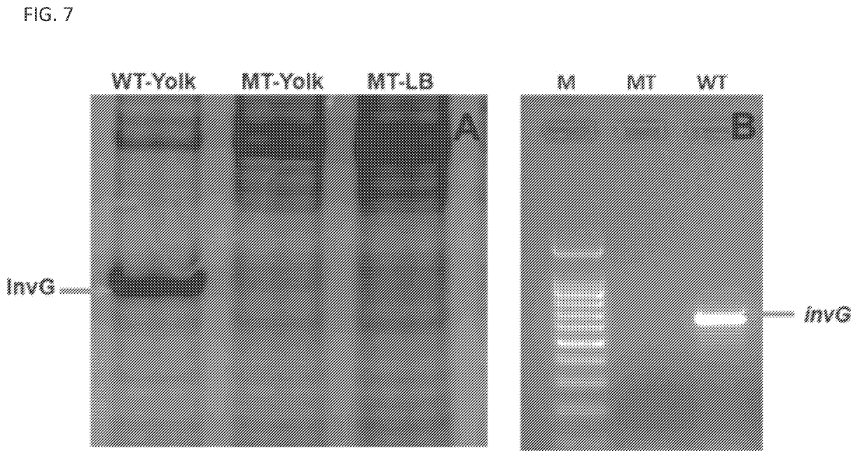

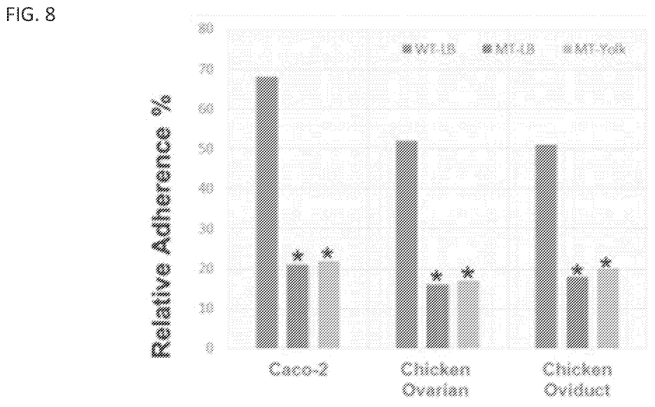

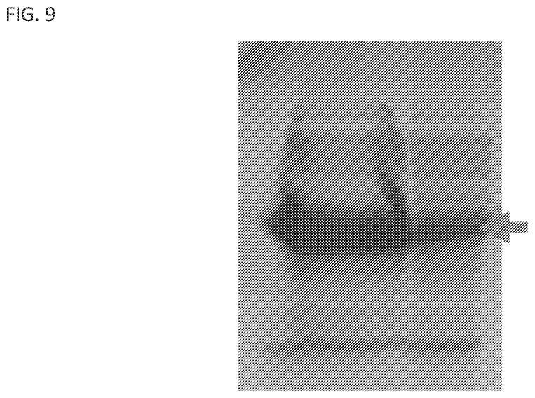

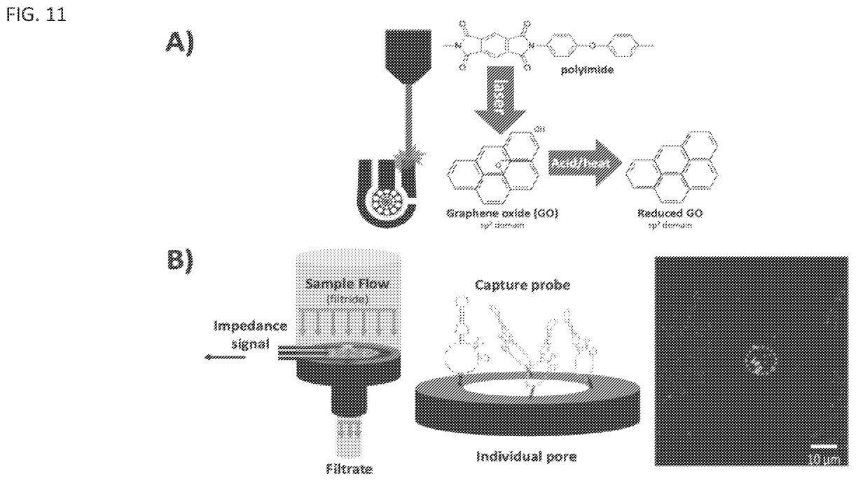

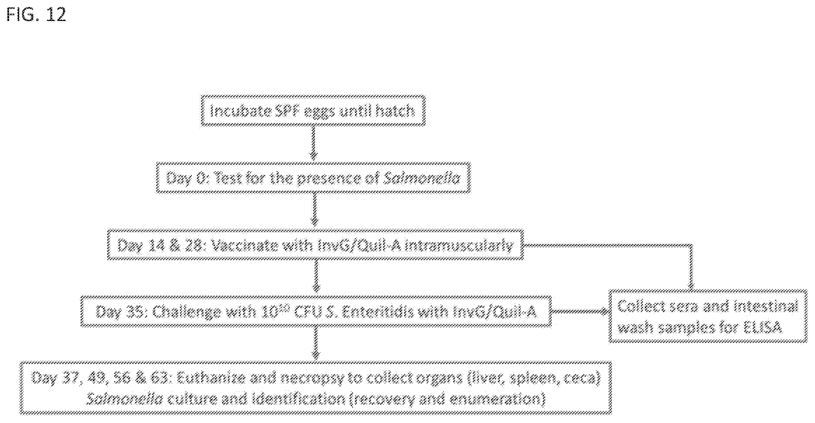

It was found that the S. Enteritidis strain SEE1 had an increase in colonization and virulence when cultivated in egg yolk compared to LB broth (FIGS. 2-5). An outer membrane protein profile and genome transcriptomic analyses revealed that InvG expression was 10-fold higher in SEE1 grown in egg yolk verses SEE1 grown in LB broth (FIGS. 6-7). SEE1 InvG null mutants had an adherent deficient phenotype compared to wild-type SEE1 (FIG. 8). Based on these experiments it was concluded that InvG contributed to the increase in colonization and virulence in SEE1 grown in egg yolk. In other experiments, a high throughput microbial test was shown to detect L. monocytogenes and E. coli in a liquid sample by using a carbon filter with microholes biofunctionalized with capture probes targeting outer membrane proteins (FIG. 11).

Based on these findings, it has been determined that immunogenic responses can be induced in poultry subjects by administering InvG to poultry subjects at risk of pathogen infection. Accordingly, in certain embodiments, disclosed are compositions comprising: S. enterica protein InvG and an adjuvant in a pharmaceutically acceptable carrier; or an avirulent organism engineered to express S. enterica protein InvG in a pharmaceutically acceptable carrier. Embodiments also include using a composition described herein to induce an immunological response in a poultry subject to protect against S. Enteritidis infection. It has been determined that pathogens can be detected by a high throughput microbial test targeting outer membrane proteins. In certain embodiments, disclosed is a method for detecting pathogens in egg yolks or other viscous liquids by utilizing InvG-T3SS as a capture probe in the high throughput microbial test.

### Overview

In the United States, two types of vaccines, S. Enteritidis (group D Salmonella) killed and S. Typhimurium (group B Salmonella) live attenuated, are being used by poultry producers in conjunction with other management practices to increase the resistance of birds against Salmonella exposure and decrease their shedding (21). Mostly, the breeder flocks, commercial layer birds, and broiler birds, to a lesser extent, are vaccinated with one or both vaccines. Numerous reports suggest that application of these vaccines have decreased the incidence of S. Enteritidis in poultry flocks (21) and the vaccines were efficacious in challenge trials (5, 21, 48), however, their efficacy in the field is still questionable (28). Killed vaccines must be administered repeatedly and they do not induce an effective cell-mediated response (6) which is required to protect poultry from Salmonella colonization. Despite many advantages of live attenuated Salmonella vaccines, major drawbacks potentially exist as well, such as the live strain persisting for long periods in poultry, their environment posing a potential threat to human health, reversion to virulence, and interference with Salmonella detection methods (7, 28, 55). Nevertheless, the protection provided by these vaccines is largely serovar specific or serogroup-specific (28). For example, these vaccines failed to provide protection against S. Braenderup (Salmonella group C1), the serovar responsible for a multistate outbreak of foodborne salmonellosis in 2017 and 2018, which was traced back to eggs (11). Although subunit vaccines are not commercially available, many proteins, including flagellar protein FliC (19, 22, 47, 59, 74), type I fimbriae, and T3SS proteins have been examined as potential vaccine candidates.

To begin the process of understanding the potential virulence gene profile of S. Enteritidis, the gene compositions of SEE1 and SEE2 was first compared with 11 selected human S. Enteritidis genome sequences from the GenBank. The predicted virulence/fitness gene profiles of SEE1 and SEE2 are based on the principle that these genes and their products may directly influence the virulence potential of S. Enteritidis either directly (e.g. effectors, toxins, and adhesins) or indirectly (e.g. nutrient acquisition systems and signaling). Based on these criteria, both SEE1 and SEE2 possess over 600 genes (13% of total genome) predicted to be involved in the virulence/fitness of S. Enteritidis and, possibly, other S. enterica serovars

The present disclosure is based on experiments, which used a mouse model of human colitis, that indicated that the mice infected with S. Enteritidis, grown in egg yolk, displayed greater illness, higher rates of colonization in the intestines and extra-intestinal organs, and higher levels of disease markers (FIGS. 2-5) than those infected with S. Enteritidis passed through mice or grown in Luria Bertani (LB) broth. These results indicated the source of S. Enteritidis infection might contribute to the overall pathogenesis of S. Enteritidis in a second host and the reservoir-pathogen dynamics might be critical for the ability of S. Enteritidis to establish colonization and virulence.

To identify the genes/proteins involved in enhanced virulence of S. Enteritidis in egg yolk, two approaches: outer membrane protein (OMP) profile analysis and whole genome transcriptomic analysis were followed. The OMP analysis identified a protein of about 60 kDa, which is present in SEE1 grown in egg yolk, but not in SEE1 grown in LB broth (FIG. 6). Mass spectrometry gave three different identities to the ˜ 60 KDa protein. Whole genome transcriptomics analysis revealed that InvG was increased by 10-fold when SEE1 was grown in egg yolk, as compared to SEE grown in LB broth. Based on the highest similarity score, the protein was identified by mass spectrometry as GroEL, InvG (a Type 3 secretion system/T3SS protein involved in invasion of intestinal cells) or PEP carboxylase (Table 1). The InvG is an outer membrane protein of the secretin family of giant β-barrel pores, conserved in the type II and Type III secretion systems, and present in all pathogenic Salmonella (34). This protein is required for the efficient entry of Salmonella into intestinal epithelial cells (35).

To determine which protein might be overexpressed when SEE1 is grown in egg yolk, invG or groEL from SEE1 were deleted to create isogenic mutants lacking the genes using Lambda Red recombination technique. This experiment confirmed the protein band identified in FIG. 6 corresponds to InvG (FIG. 7). As shown in FIG. 8, the mutant strain grown in LB broth or egg yolk (MT-LB and MT-yolk) demonstrated an adherent deficient phenotype on Caco-2, chicken ovarian epithelial, and chicken oviduct epithelial cells, as compared to the wild-type strain grown in egg yolk (WT-Yolk).

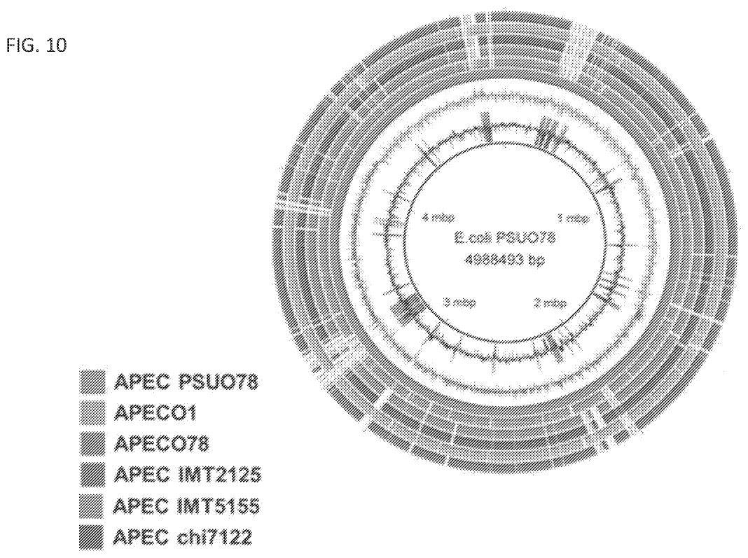

In one embodiment, disclosed is a vector strain of APEC (PSUO78) sequenced and deposited in the GenBank under the accession number CP012112.1. This APEC strain was isolated from the oviduct of a hen having E. coli-induced salpingitis/peritonitis (FIG. 10). An aroA mutant of this strain PSUO78/aroA is used as the vaccine vector strain. It has been demonstrated that this vaccine is safe, immunogenic and has the ability to protect chickens from colibacillosis, including salpingitis/peritonitis infection, when administered via oral route.

In a further embodiment, disclosed is a purified S. Enteritidis InvG as a 6×His-tagged protein collected under denaturing conditions using nickel charged resins (Qiagen Inc., Germantown, MD) according to manufacturer instructions. The purity is confirmed by running the purified protein on an SDS-PAGE followed by Coomassie blue staining.

In one embodiment, disclosed is an APEC-vectored vaccine expressing the InvG of SEE1, due to the limitations in administering a subunit vaccine to poultry under field conditions and inferior protection provided by subunit vaccines. Using E. coli as a vector obviates the concerns of using live Salmonella, such as live Salmonella persisting for long periods in poultry or their environment posing a potential threat to human health, reversion to virulence, and interference with Salmonella detection methods. Specifically, an aroA mutant of APEC strain PSUO78 (PSUO78-aroA strain described above) is used as the vector. The aroA mutant is constructed as previously described by Kariyawasam et al (36). The size of and location of the deletions for the mutants is provided in Table 1 below. This APEC vectored vaccine protects poultry from E. coli-associated peritonitis in addition to reducing Salmonella colonization and shedding. To construct the vectored vaccine expressing Salmonella InvG antigen, the SEE1 antigen is first cloned into the expression vector pBR322 (Roche). Then, the cloned is introduced into the PSU-O78-aroA strain.

TABLE 1
(note size of aroA deletion can be 692 bp)

| | | Restriction enzyme | Size of | Size of the |
|---|---|---|---|---|
| | | used to make | the gene | deletion |
| Gene | Primer sequence^{A} | the deletion^{B} | (bp) | (bp) |
| galE | 5′-CCATGGTACCGTTACGCGCCAGTTCAGTT-3′ | HaeII (159, 618) | 1016 | 459 |
| | 5′-GGCCGAGCTCTTGGAAGTCATACCTGTGTG-3′ | | | |
| purA | 5′-GCGCGGTACCTACTCTCGTAATCAACGGTG-3′ | HpaI (396, 1385) | 2726 | 989 |
| | 5′-GGCCGAGCTCAGTGAAGACCAGTTGACCAT-3′ | | | |
| aroA | 5′-CCATGGTACCTCGTGTCGATGGCACTATTA-3′ | BglI (259, 951) | 1283 | 672 |
| | 5′-GGCCGAGCTCTCAAGAATCGTCACTGGTGT-3′ | | | |

^{A}Primer sequences were based on sequences in GenBank AE000178, J04199, and AE000193 for galE, purA,
and aroa genes, respectively. Each forward and reverse primer has KpnI and SacI restriction sites, respectively at the 5′ ends.
^{B}Positions of the restriction site on the amplified PCR product are indicated within the brackets.

### Pharmaceutical Compositions

In other embodiments, pharmaceutical compositions are provided that comprise at least one immunogenic polypeptide comprising at least one S. enterica protein that is required for or involved in avian transmission of S. enterica. In some embodiments, the S. enterica protein required for or involved in avian transmission of S. enterica has an amino acid sequence set forth in SEQ ID NO:2 or an immunogenic fragment or variant of any thereof, and a non-naturally occurring pharmaceutically acceptable carrier.

With respect to the amino acid sequences for the various full-length polypeptides, variants include those polypeptides that are derived from the native polypeptides by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Such variants may result from, for example, genetic polymorphism or from human manipulation. Methods for mutagenesis and nucleotide sequence alterations are well known in the art. See, for example, Walker and Gaastra, eds. (1983) Techniques in Molecular Biology Cells engineered to express protein.

In certain embodiments, the pharmaceutical composition comprises a microorganism engineered to be avirulent and to be a vector that includes a polynucleotide sequence encoding a S. enterica InvG protein according to SEQ ID NO: 2, or immunogenic fragment or variant thereof, and optionally, a non-naturally occurring pharmaceutically acceptable carrier. The InvG antigen is cloned from SEE1 into expression vector pBR322. The expression vector is introduced to avirulent E. coli using methods know in the arts. In some embodiments, the microorganism is a mutant aroA, with an amino acid sequence set forth as SEQ ID NO:3, E. coli strain PSUO78.

The presently disclosed pharmaceutical compositions comprise an immunogenic polypeptide and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. In certain embodiments, the presently disclosed pharmaceutical compositions comprise a non-naturally occurring pharmaceutically acceptable carrier. That is, a carrier that is not normally found in nature or not normally found in nature in combination with the immunogenic polypeptide.

In one embodiment, the pharmaceutical composition is in bulk or in unit dosage form. The unit dosage form is any of a variety of forms, including, for example, a capsule, a tablet, or a vial. The quantity of active ingredient in a unit dose of composition is an effective amount and is varied according to the particular treatment involved.

Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as drinking water or spray. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide.

### Methods of Administration

In certain embodiments, the compositions are administered intramuscularly to poultry subjects. Where the subject is a layer hen, the composition of this invention would be administered once the subject has reached a steady state of laying or 26 weeks of age. The composition would be administered every 14 days until 3 or 4 doses have been administered. Where the subject is a newly hatch chick, the composition of this invention would be administered at 14 days of age and again at 28 days of age. For other poultry species, the optimal range of days for intramuscular administration of a composition of this invention can be determined according to methods well known in the art.

In other embodiments, the compositions are administered orally to poultry subjects by methods of mass administration. The delivery of the compositions can be via drinking water, spray, or feed. Where the subject is a layer hen, the composition of this invention would be administered once the subject has reached a steady state of laying or 26 weeks of age. The composition would be administered twice 14 days apart. Where the subject is a newly hatch chick, the composition of this invention would be administered at 14 days of age and again at 28 days of age. For other poultry species, the optimal range of days for oral administration of a composition of this invention can be determined according to methods well known in the art.

In yet further embodiments, the compositions are administered in the final quarter of egg incubation of the poultry subject. Where the subject is a chicken, the final quarter to administer the composition of this invention in ovo would be during the period from day 15 through day 20 of fertile egg incubation, and in particular embodiments, the composition can be administered on day 18 or day 19 of incubation. When the subject is a turkey, the final quarter for administration would be during the period from day 21 through day 28 of incubation and in particular embodiments, the compositions can be administered on day 24 or day 25 of incubation. In other embodiments wherein the subject is a goose, the final quarter of administration would be during the period from day 23 through day 31 of incubation and in particular embodiments, the compositions can be administered on day 28 or day 29 of incubation. In further embodiments wherein the subject is a duck, the final quarter of administration would be during the period from day 21 through day 28 of incubation and in particular embodiments, the compositions can be administered on day 25 or day 26 of incubation.

For other poultry species, the final quarter of incubation and thus the optimal range of days for in ovo administration of a composition of this invention can be determined according to methods well known in the art. For example, a muscovy duck has an incubation period in the range of 33-35 days, a ringneck pheasant has an incubation period of 23-24 days, a Japanese quail has an incubation period of 17-18 days, a bobwhite quail has an incubation period of 23 days, a chuckar partridge has an incubation period of 22-23 days, a guinea has an incubation period of 26-28 days and a peafowl has an incubation period of 28 days.

### Sequences

As described herein, embodiments pertain to an InvG protein or an immunogenic fragment or variant thereof. In one embodiment, the nucleic acid encoding an InvG protein is SEQ ID NO:1.

| SEQ ID NO: 1 | |
|---|---|
| agtaaattaa cggaggactg gagataggtc tatttgtgct tgggtaaaga cattgaatag | 60 |
| caggggtgag gtccgtggcg aaacgaagtc ttataacaaa tggctaagcg accgtaggcc | 120 |
| gcaattgccc agctgttaaa gaaaaccaag ttagtacttg tgtgcttgtt gtaatgaaaa | 180 |
| taagaatgac cttatcgcct tgtccgatgg ttattcgcca ttaaacggat ttttgcctta | 240 |
| tgaaacctgt catagccata aacgtagggc acatattggt ggctggtcgt ttgaaaaagg | 300 |
| cacgccgtga gagcgttatc acgattaatt gcaagcgggc tgaagcccat tgcgtagatg | 360 |
| cctccaccat catagcctaa cgccgcagaa tagcaacggt agaagttaca ggtcgctgta | 420 |
| aagatagact ggtagacgcc tttttgcccc gtcgtgagcc tagtaacaag gcatacaatg | 480 |
| tacgagttcg cggtgtaatg caaggggtta gtcgaaccac attttgcaag acaacaatag | 540 |
| tttttatcgc ccttgtaaaa ggacccagtc attatggccc gcactttggt ggcagcggac | 600 |
| aaagaagaga agattgcgta actggcgccg ctacttagct gacggtagct cccatgaata | 660 |
| acttctgacc aaattactgt gcggttcaaa caggggttat cattacgacg gcgaggtact | 720 |
| tcacgggttt gcgaggtcta gcgaaaataa ttctagctgt taggtgtccc tattaagatg | 780 |
| cactgcaaac cgctgtaggt cgcgaaattg gtcgtaaagc tattttacgt ggacgagtcg | 840 |
| gcaagggaaa tgattgtttg acaaccatag gcctatccgg tgctaaaatt ataacgggcg | 900 |
| gcgccgtaaa acgaaatttc gaaggacgtc tgagtacggt ggccgtatta aacgacgaaa | 960 |
| tggaaaaaga ggtaagcgac ttttgcgacc gtagcggccc ccaagcgatg actgttataa | 1020 |
| tggatttcca acgagaagag ggacgttgtt ggaaagttac cggcaccgtt atggccccta | 1080 |
| ttggtaaaag actagcgcgt ctaatatcca tgctagcggg tgcttccata acaagtctgc | 1140 |
| gtagtgggga taaaagactg cagggtcgag ttatggtagc aaaacgaaca ggtagtacca | 1200 |
| ccgccgcaac tggtggtata gttgtatctg cccaccggga ctttgtatct tacaaggaaa | 1260 |
| tgccaatagc ggtgcatcgc ccattaaaaa caatatattt ggactcgcaa aatcttttaa | 1320 |
| caacttgagt aactcactct gcaacgcatt tctttggtgc cgtaacgcgt aaagtgaccg | 1380 |
| cagtatttat atctatcgga cgggtagctt tatggtttag tcggggtcaa catccctttc | 1440 |
| gaagaggtca ttacgcaatc ctagcacttt gagtttcaac gggcattaaa aaaaagcacg | 1500 |
| gcggtaaaac gattgttact gtccgaggaa atcgacatcg cggtaccgta gctttttaca | 1560 |
| ggcgtccgat agcagaaagc gttgtttggg tgaagggcaa tgtccataaa aaagtgatct | 1620 |
| tattggtcca cattggtctt gttcgcgccg tgtacggtcg tgagaccggt tttcttatac | 1680 |
| acagaagta | 1689 |

In a further embodiment, the InvG protein comprises SEQ ID NO:2 or immunogenic fragments or variants thereof.

```
SEQ ID NO. 2: amino acid sequence of S. enterica InvG protein
Met Lys Thr His Ile Leu Leu Ala Arg Val Leu Ala Cys Ala Ala Leu
1 5 10 15
Val Leu Val Thr Pro Gly Tyr Ser Ser Glu Lys Ile Pro Val Thr Gly
                        20 25 30
Ser Gly Phe Val Ala Lys Asp Asp Ser Leu Arg Thr Phe Phe Asp Ala
                35 40 45
Met Ala Leu Gln Leu Lys Glu Pro Val Ile Val Ser Lys Met Ala Ala
        50 55 60
Arg Lys Lys Ile Thr Gly Asn Phe Glu Phe His Asp Pro Asn Ala Leu
65 70 75 80
Leu Glu Lys Leu Ser Leu Gln Leu Gly Leu Ile Trp Tyr Phe Asp Gly
                                85 90 95
Gln Ala Ile Tyr Ile Tyr Asp Ala Ser Glu Met Arg Asn Ala Val Val
                        100 105 110
Ser Leu Arg Asn Val Ser Leu Asn Glu Phe Asn Asn Phe Leu Lys Arg
                115 120 125
Ser Gly Leu Tyr Asn Lys Asn Tyr Pro Leu Arg Gly Asp Asn Arg Lys
        130 135 140
Gly Thr Phe Tyr Val Ser Gly Pro Pro Val Tyr Val Asp Met Val Val
145 150 155 160
Asn Ala Ala Thr Met Met Asp Lys Gln Asn Asp Gly Ile Glu Leu Gly
                                165 170 175
Arg Gln Lys Ile Gly Val Met Arg Leu Asn Asn Thr Phe Val Gly Asp
                        180 185 190
Arg Thr Tyr Asn Leu Arg Asp Gln Lys Met Val Ile Pro Gly Ile Ala
                195 200 205
Thr Ala Ile Glu Arg Leu Leu Gln Gly Glu Glu Gln Pro Leu Gly Asn
        210 215 220
Ile Val Ser Ser Glu Pro Pro Ala Met Pro Ala Phe Ser Ala Asn Gly
225 230 235 240
Glu Lys Gly Lys Ala Ala Asn Tyr Ala Gly Gly Met Ser Leu Gln Glu
                                245 250 255
Ala Leu Lys Gln Asn Ala Ala Ala Gly Asn Ile Lys Ile Val Ala Tyr
                        260 265 270
Pro Asp Thr Asn Ser Leu Leu Val Lys Gly Thr Ala Glu Gln Val His
                275 280 285
Phe Ile Glu Met Leu Val Lys Ala Leu Asp Val Ala Lys Arg His Val
        290 295 300
Glu Leu Ser Leu Trp Ile Val Asp Leu Asn Lys Ser Asp Leu Glu Arg
305 310 315 320
Leu Gly Thr Ser Trp Ser Gly Ser Ile Thr Ile Gly Asp Lys Leu Gly
                                325 330 335
Val Ser Leu Asn Gln Ser Ser Ile Ser Thr Leu Asp Gly Ser Arg Phe
                        340 345 350
Ile Ala Ala Val Asn Ala Leu Glu Glu Lys Lys Gln Ala Thr Val Val
                355 360 365
Ser Arg Pro Val Leu Leu Thr Gln Glu Asn Val Pro Ala Ile Phe Asp
        370 375 380
Asn Asn Arg Thr Phe Tyr Thr Lys Leu Ile Gly Glu Arg Asn Val Ala
385 390 395 400
Leu Glu His Val Thr Tyr Gly Thr Met Ile Arg Val Leu Pro Arg Phe
                                405 410 415
Ser Ala Asp Gly Gln Ile Glu Met Ser Leu Asp Ile Glu Asp Gly Asn
                        420 425 430
Asp Lys Thr Pro Gln Ser Asp Thr Thr Thr Ser Val Asp Ala Leu Pro
                435 440 445
Glu Val Gly Arg Thr Leu Ile Ser Thr Ile Ala Arg Val Pro His Gly
        450 455 460
Lys Ser Leu Leu Val Gly Gly Tyr Thr Arg Asp Ala Asn Thr Asp Thr
465 470 475 480
Val Gln Ser Ile Pro Phe Leu Gly Lys Leu Pro Leu Ile Gly Ser Leu
                                485 490 495
Phe Arg Tyr Ser Ser Lys Asn Lys Ser Asn Val Val Arg Val Phe Met
                        500 505 510
Ile Glu Pro Lys Glu Ile Val Asp Pro Leu Thr Pro Asp Ala Ser Glu
                515 520 525
Ser Val Asn Asn Ile Leu Lys Gln Ser Gly Ala Trp Ser Gly Asp Asp
        530 535 540
Lys Leu Gln Lys Trp Val Arg Val Tyr Leu Asp Arg Gly Gln Glu Ala
545 550 555 560
Ile Lys
```

As noted, immunogenic fragments and variants of S. enterica InvG protein are described and may be administered to a poultry subject. Such immunogenic fragments can comprise at least about 5, at least about 10, at least about 15, at least about 20, at least about 50, at least about 60, at least about 80, at least about 100, at least about 150, at least about 200, at least about 300, at least about 400, at least about 500, at least about 600, at least about 700, at least about 800, at least about 900, at least about 1,000 contiguous amino acid residues or up to the entire contiguous amino acid residues of the InvG protein (e.g. SEQ ID NO:2).

In a further embodiment, disclosed is a nucleic acid that encodes a mutant aroA protein. In a specific embodiment, the aroA protein is SEQ ID NO: 3 or immunogenic fragments or variants thereof.

| SEQ ID NO: 3 | |
|---|---|
| atggaatccc tgacgttaca acccatcgct cgtgtcgatg gcactattaa tctgcccggt | 60 |
| tccaagagcg tttctaaccg cgctttattg ctggcggcat tagcacacgg caaaacagta | 120 |
| ttaaccaatc tgctggatag cgatgacgtg cgccatatgc tgaatgcatt aacagcgtta | 180 |
| ggggtaagct atacgctttc agccgatcgt acgcgttgcg aaattatcgg taacggcggt | 240 |
| ccattacacg cagaaggtga ttgccacggc ggcgttattt gcaaaaggca ccaccacgct | 300 |
| gcgcaatatc tataactggc gtgttaaaga gaccgatcgc ctgtttgcga tggcaacaga | 360 |
| actgcgtaaa gtcggcgcgg aagtggaaga ggggcacgat tacattcgta tcactcctcc | 420 |
| ggaaaaactg aactttgccg agatcgcgac atacaatgat caccggatgg cgatgtgttt | 480 |
| ctcgctggtg gcgttgtcag atacaccagt gacgattctt gatcccaaat gcacggccaa | 540 |
| aacatttccg gattatttcg agcagctggc gcggattagc caggcagcct ga | |

Also disclosed is a nucleic acid sequence that encode an expression vector pBR322. In a specific embodiment, the expression vector pBR322 comprises the nucleic acid sequence of SEQ ID NO: 4.

| SEQ ID NO: 4 | |
|---|---|
| ttctcatgtt tgacagctta tcatcgataa gctttaatgc ggtagtttat cacagttaaa | 60 |
| ttgctaacgc agtcaggcac cgtgtatgaa atctaacaat gcgctcatcg tcatcctcgg | 120 |
| caccgtcacc ctggatgctg taggcatagg cttggttatg ccggtactgc cgggcctctt | 180 |
| gcgggatatc gtccattccg acagcatcgc cagtcactat ggcgtgctgc tagcgctata | 240 |
| tgcgttgatg caatttctat gcgcacccgt tctcggagca ctgtccgacc gctttggccg | 300 |
| ccgcccagtc ctgctcgctt cgctacttgg agccactatc gactacgcga tcatggcgac | 360 |
| cacacccgtc ctgtggatcc tctacgccgg acgcatcgtg gccggcatca ccggcgccac | 420 |
| aggtgcggtt gctggcgcct atatcgccga catcaccgat ggggaagatc gggctcgcca | 480 |
| cttcgggctc atgagcgctt gtttcggcgt gggtatggtg gcaggccccg tggccggggg | 540 |
| actgttgggc gccatctcct tgcatgcacc attccttgcg gcggcggtgc tcaacggcct | 600 |
| caacctacta ctgggctgct tcctaatgca ggagtcgcat aagggagagc gtcgaccgat | 660 |
| gcccttgaga gccttcaacc cagtcagctc cttccggtgg gcgcggggca tgactatcgt | 720 |
| cgccgcactt atgactgtct tctttatcat gcaactcgta ggacaggtgc cggcagcgct | 780 |
| ctgggtcatt ttcggcgagg accgctttcg ctggagcgcg acgatgatcg gcctgtcgct | 840 |
| tgcggtattc ggaatcttgc acgccctcgc tcaagccttc gtcactggtc ccgccaccaa | 900 |
| acgtttcggc gagaagcagg ccattatcgc cggcatggcg gccgacgcgc tgggctacgt | 960 |
| cttgctggcg ttcgcgacgc gaggctggat ggccttcccc attatgattc ttctcgcttc | 1020 |
| cggcggcatc gggatgcccg cgttgcaggc catgctgtcc aggcaggtag atgacgacca | 1080 |
| tcagggacag cttcaaggat cgctcgcggc tcttaccagc ctaacttcga tcactggacc | 1140 |
| gctgatcgtc acggcgattt atgccgcctc ggcgagcaca tggaacgggt tggcatggat | 1200 |
| tgtaggcgcc gccctatacc ttgtctgcct ccccgcgttg cgtcgcggtg catggagccg | 1260 |
| ggccacctcg acctgaatgg aagccggcgg cacctcgcta acggattcac cactccaaga | 1320 |
| attggagcca atcaattctt gcggagaact gtgaatgcgc aaaccaaccc ttggcagaac | 1380 |
| atatccatcg cgtccgccat ctccagcagc cgcacgcggc gcatctcggg cagcgttggg | 1440 |
| tcctggccac gggtgcgcat gatcgtgctc ctgtcgttga ggacccggct aggctggcgg | 1500 |
| ggttgcctta ctggttagca gaatgaatca ccgatacgcg agcgaacgtg aagcgactgc | 1560 |
| tgctgcaaaa cgtctgcgac ctgagcaaca acatgaatgg tcttcggttt ccgtgtttcg | 1620 |
| taaagtctgg aaacgcggaa gtcagcgccc tgcaccatta tgttccggat ctgcatcgca | 1680 |
| ggatgctgct ggctaccctg tggaacacct acatctgtat taacgaagcg ctggcattga | 1740 |
| ccctgagtga tttttctctg gtcccgccgc atccataccg ccagttgttt accctcacaa | 1800 |
| cgttccagta accgggcatg ttcatcatca gtaacccgta tcgtgagcat cctctctcgt | 1860 |
| ttcatcggta tcattacccc catgaacaga aatccccctt acacggaggc atcagtgacc | 1920 |
| aaacaggaaa aaaccgccct taacatggcc cgctttatca gaagccagac attaacgctt | 1980 |
| ctggagaaac tcaacgagct ggacgcggat gaacaggcag acatctgtga atcgcttcac | 2040 |
| gaccacgctg atgagcttta ccgcagctgc ctcgcgcgtt tcggtgatga cggtgaaaac | 2100 |
| ctctgacaca tgcagctccc ggagacggtc acagcttgtc tgtaagcgga tgccgggagc | 2160 |
| agacaagccc gtcagggcgc gtcagcgggt gttggcgggt gtcggggcgc agccatgacc | 2220 |
| cagtcacgta gcgatagcgg agtgtatact ggcttaacta tgcggcatca gagcagattg | 2280 |
| tactgagagt gcaccatatg cggtgtgaaa taccgcacag atgcgtaagg agaaaatacc | 2340 |
| gcatcaggcg ctcttccgct tcctcgctca ctgactcgct gcgctcggtc gttcggctgc | 2400 |
| ggcgagcggt atcagctcac tcaaaggcgg taatacggtt atccacagaa tcaggggata | 2460 |
| acgcaggaaa gaacatgtga gcadaaggcc agcaaaaggc caggaaccgt aaaaaggccg | 2520 |
| cgttgctggc gtttttccat aggctccgcc cccctgacga gcatcacaaa aatcgacgct | 2580 |
| caagtcagag gtggcgaaac ccgacaggac tataaagata ccaggcgttt ccccctggaa | 2640 |
| gctccctcgt gcgctctcct gttccgaccc tgccgcttac cggatacctg tccgcctttc | 2700 |
| tcccttcggg aagcgtggcg ctttctcata gctcacgctg taggtatctc agttcggtgt | 2760 |
| aggtcgttcg ctccaagctg ggctgtgtgc acgaaccccc cgttcagccc gaccgctgcg | 2820 |
| ccttatccgg taactatcgt cttgagtcca acccggtaag acacgactta tcgccactgg | 2880 |
| cagcagccac tggtaacagg attagcagag cgaggtatgt aggcggtgct acagagttct | 2940 |
| tgaagtggtg gcctaactac ggctacacta gaaggacagt atttggtatc tgcgctctgc | 3000 |
| tgaagccagt taccttcgga aaaagagttg gtagctcttg atccggcaaa caaaccaccg | 3060 |
| ctggtagcgg tggttttttt gtttgcaagc agcagattac gcgcagaaaa aaaggatctc | 3120 |
| aagaagatcc tttgatcttt tctacggggt ctgacgctca gtggaacgaa aactcacgtt | 3180 |
| aagggatttt ggtcatgaga ttatcaaaaa ggatcttcac ctagatcctt ttaaattaaa | 3240 |
| aatgaagttt taaatcaatc taaagtatat atgagtaaac ttggtctgac agttaccaat | 3300 |
| gcttaatcag tgaggcacct atctcagcga tctgtctatt tcgttcatcc atagttgcct | 3360 |
| gactccccgt cgtgtagata actacgatac gggagggctt accatctggc cccagtgctg | 3420 |
| caatgatacc gcgagaccca cgctcaccgg ctccagattt atcagcaata aaccagccag | 3480 |
| ccggaagggc cgagcgcaga agtggtcctg caactttatc cgcctccatc cagtctatta | 3540 |
| attgttgccg ggaagctaga gtaagtagtt cgccagttaa tagtttgcgc aacgttgttg | 3600 |
| ccattgctgc aggcatcgtg gtgtcacgct cgtcgtttgg tatggcttca ttcagctccg | 3660 |
| gttcccaacg atcaaggcga gttacatgat cccccatgtt gtgcaaaaaa gcggttagct | 3720 |
| ccttcggtcc tccgatcgtt gtcagaagta agttggccgc agtgttatca ctcatggtta | 3780 |
| tggcagcact gcataattct cttactgtca tgccatccgt aagatgcttt tctgtgactg | 3840 |
| gtgagtactc aaccaagtca ttctgagaat agtgtatgcg gcgaccgagt tgctcttgcc | 3900 |
| cggcgtcaac acgggataat accgcgccac atagcagaac tttaaaagtg ctcatcattg | 3960 |
| gaaaacgttc ttcggggcga aaactctcaa ggatcttacc gctgttgaga tccagttcga | 4020 |
| tgtaacccac tcgtgcaccc aactgatctt cagcatcttt tactttcacc agcgtttctg | 4080 |
| ggtgagcaaa aacaggaagg caaaatgccg caaaaaaggg aataagggcg acacggaaat | 4140 |
| gttgaatact catactcttc ctttttcaat attattgaag catttatcag ggttattgtc | 4200 |
| tcatgagcgg atacatattt gaatgtattt agaaaaataa acaaataggg gttccgcgca | 4260 |
| catttccccg aaaagtgcca cctgacgtct aagaaaccat tattatcatg acattaacct | 4320 |
| ataaaaatag gcgtatcacg aggccctttc gtcttcaaga a | 4361 |

### Methods of Detecting Presence of Pathogens

Although Salmonella culture and identification is the gold standard method of detecting Salmonella in the bird, environment, and food samples (24, 75), some molecular-based and immunological diagnostic tools have also been approved by FDA and NPIP recently (25). However, all these tests still require a pre-enrichment step for improved detection resolution, making the tests more laborious and/or time consuming. Overcoming these obstacles continues to be one of the major thrusts for further development and refinement of Salmonella detection methods.

In one embodiment, disclosed is a method for multiple high throughput microbial tests for rapid analysis of large sample volumes in agricultural testing for Salmonella, based on aptamers targeting outer membrane proteins. The initial test involved flow of 10 ml through a microfluidic channel in the presence of bacteria-sized ferromagnetic discs (9, 10), which may be used in initial screening studies. To extend this test for high throughput applications, an electrically-active carbon filter was developed that can be incorporated with any laboratory test using vacuum-driven flow or pressure-driven flow (FIG. 11). Emulsion-coated cellulose paper or nitrocellulose membranes are the backing material for the flexible carbon circuit.

The electrically active filter is prepared by laser carbonization, using methods published previously (3, 27, 70) (FIG. 11A). After forming the base circuit pattern with interdigitated electrodes, an Nd:Yag laser is used to perforate the carbon circuit (10 μm holes). The perforated holes are then metallized with platinum or gold (70), and these metal-coated microholes are then biofunctionalized with capture probes. The metal-coated carbon surface is functionalized with InvG-T3SS by first tholating the protein using Traut's reagent, and then determining the optimum adsorption for maximum surface coverage. To date, antibodies, aptamers, lectins, and oligopeptides that target extracellular membrane proteins have been tested on L. monocytogenes or E. coli. The number of perforations is adjusted to the sample throughput needs, and sample is driven through the orifice by either vacuum or positive pressure. To date, as few as 20 CFU/L E. coli have been detected in irrigation water, which allows rapid screening with no preenrichment or labeling. Electrochemical impedance spectroscopy is used for real time analysis of cell capture, and all measurements are validated with AOAC methods (PCR and cell culture).

Disclosed embodiments provide a method for measuring pathogen presence in food or other samples. The samples are viscous liquids for example broths, stocks, milk, creams, or diluted egg yolk. Various perforation geometries in the carbon circuit are used to optimize the flow rate for test samples while simultaneously limiting dead zones on the filter. Real time analysis of viscous fluids (egg yolk, creams, etc.) is a major step forward in food safety analysis, as current methods rely on standard cell culture after significant dilution and pre-enrichment. The conventional methods used today are expensive, time-consuming, and require trained expertise for accuracy.

### EXAMPLES

### Example 1: The Immunogenicity and Efficacy of InvG Protein of Salmonella Enteritidis Against a Challenge of S. Enteritidis in Chickens

In this example, immunogenicity of the protein and efficacy against a homologous challenge is assessed. Newly hatched chicks are randomly divided into three groups of 20 chickens. Chickens in group 1 are vaccinated intramuscularly with 50 μg of InvG and 50 μg of Quil-A at days 14 and 28 days of age. The chickens are challenged with 1010 CFU of S. Enteritidis strain SEE1 in 0.5 ml of phosphate buffered saline administered orally at 35 days of age. Five birds from each group are sacrificed on day 2, 7, 14, and 21 post-challenge. Liver, spleen, and cecal contents are collected at necropsy for Salmonella culture and enumeration.

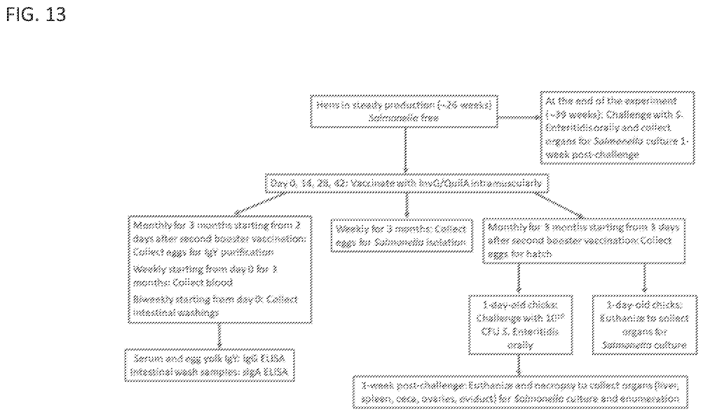

Chickens in group 2 serve as the negative control group and receive PBS intramuscularly on day 14 and 28, and orally on day 35. Chickens in group 3 serve as the positive control group and receive PBS intramuscularly on day 14 and 28, and S. Enteritidis orally on day 35. Blood is collected prior to immunization and at the time of euthanasia (day 2, 7, 14, and 21 post-challenge) to measure anti-InvG IgG titers by ELISA (FIG. 13). To measure sIgA titers, intestinal washing samples (preimmunization) are collected at day 14, 28, and 35.

### Example 2: Antibodies Against InvG can Transfer Passively from Vaccinated Hens to Progeny Chickens Thus Providing Protection

In this example, layer chickens are vaccinated with InvG, and the eggs laid by the vaccinated hens and their progeny chicks are monitored for anti-InvG and sIgA antibodies. One-day-old progeny chicks are also challenged with S. Enteritidis to assess the protection provided by passively transferred antibodies.

Chicken homologous Salmonella challenge. For this experiment, two groups (20 birds/group) of laying hens are used. Each group is housed with 2 male chickens to obtain fertile eggs. After hens in both groups reached a steady state of laying (˜26 weeks), hens in one group are immunized three times with 50 μg of InvG/Quil-A three weeks apart. The hens in the other group receive PBS and serve as the negative control. To ensure that the chickens are Salmonella free, another five birds are euthanized to collect ceca, ovaries, liver, and spleen for bacterial culture prior to the experiment. Groups treated with PBS (intramuscular)/PBS (oral) and PBS (intramuscular)/Salmonella (oral) serve as negative and positive controls, respectively. Blood and intestinal washings are collected prior to immunization and weekly thereafter until euthanasia to measure anti-InvG and anti-sIgA titers by ELISA. Eggs are collected daily for 3 months after the last booster vaccination and used for hatching, purifying IgY, or microbiological testing alternatively at 3-day intervals (e.g. day 1 post-immunization for hatching; day 2 post-immunization for purifying IgY; day 3 postimmunization for Salmonella culture). For IgY purification and Salmonella culture, eggs are tested in pools of 5 eggs. Hens are bled every week until the end of the experiment to measure anti-InvG titers. Intestinal washings are also collected every two weeks for sIgA ELISA. Half of the hatched chicks are euthanized on day 1 to collect organs (liver, spleen and ceca) for Salmonella culture. The other half are challenged orally with 1010 CFU of S. Enteritidis strain SEE1 as mentioned above. One week after challenge, organs are collected for Salmonella culture. At the end of the experiment, adult hens are challenged with 1010 CFU of S. Enteritidis and organs (liver, spleen, ovaries, oviduct, and ceca) are collected for Salmonella culture, identification, and enumeration (FIG. 13).

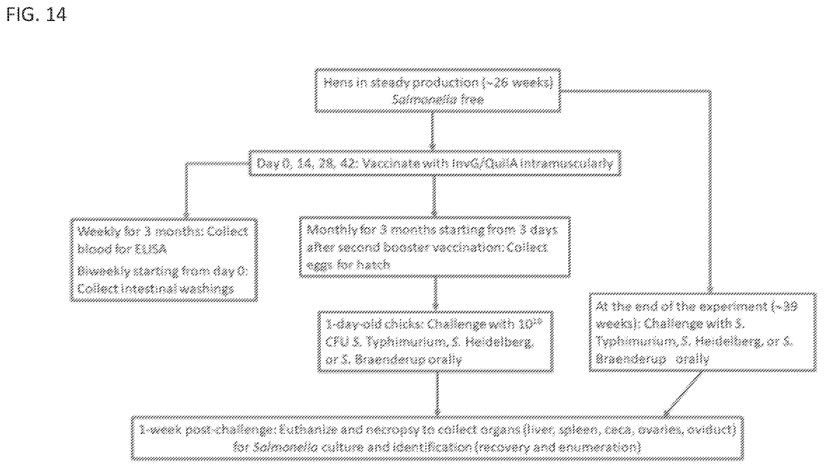

Chicken heterologous Salmonella challenge. Two groups of chickens (30 chickens) are kept with male birds to obtain fertile eggs. One group is vaccinated three times with InvG/Quil-A as described under the example 2—homologue challenge. Eggs laid on day 7/8, 14/15, 21/22, and 28/29 are hatched to obtain chicks for challenge experiments. Chicks are divided into four groups at each time point (day 7/8, 14/15, 21/22, or 28/29). Groups 1, 2, and 3 are challenged with 1010 CFU of S. Typhimurium, S. Heidelberg, or S. Braenderup, respectively. Group 4 receive PBS and serve as the placebo control (FIG. 14).

### Example 3: An E. coli-Vectored Vaccine Expressing InvG to Minimize Colonization of Chicken Intestines and Ovaries by Nontyphoidal Salmonella

Chicken challenge experimental approach will be similar to the approach described under the example 2 (FIGS. 13 and 14) except that the chickens are vaccinated with 1×10⁷ CFU/ml of bacteria in PBS inoculated orally twice (day 14 and 28) in place of four-dose recombinant protein antigen vaccination regimen. Challenging chickens with S. Enteritidis, S. Typhimurium, S. Heidelberg, and S. Branenderup, measurement of anti-InvG antibodies in the chicken sera, intestinal washings, and eggs, and collection of tissues, bacterial culture is performed as described under the example 2. Appropriate negative and positive controls are used.

### REFERENCES

1. The New York Times. U.S. Rejected Hen Vaccine Despite British Success https://www.nytimes.com/2010/08/25/business/25vaccine.html.
2. Outbreaks of Salmonella serotype Enteritidis infection associated with eating raw or undercooked shell eggs—United States, 1996-1998. MMWR Morb Mortal Wkly Rep 49:73-79. 2000.
3. Abdelbasir, S. M. S. M. E.-S., V. L. Morgan, H. Schmidt, L. M. Casso-Hartmann, D. C. Vanegas, I. Velez-Torres, E. S. McLamore Graphene-anchored cuprous oxide nanoparticles from waste electric cables for electrochemical sensing. ACS Sustainable Chemical Engineering, 6 (9), pp 12176-12186. DOI: 10.1021/acssuschemeng.8b02510. 2018.
4. Antunes, P., J. Mourao, J. Campos, and L. Peixe. Salmonellosis: the role of poultry meat. Clin Microbiol Infect 22:110-121. 2016.
5. Atterbury, R. J., R. H. Davies, J. J. Carrique-Mas, V. Morris, D. Harrison, V. Tucker, and V. M. Allen. Effect of delivery method on the efficacy of Salmonella vaccination in chickens. Vet Rec 167:161-164. 2010.
6. Babu, U., M. Scott, M. J. Myers, M. Okamura, D. Gaines, H. F. Yancy, H. Lillehoj, R. A. Heckert, and R. B. Raybourne. Effects of live attenuated and killed Salmonella vaccine on Tlymphocyte mediated immunity in laying hens. Vet Immunol Immunopathol 91:39-44. 2003.
7. Barrow, P. A. Salmonella infections: immune and non-immune protection with vaccines. Avian Pathol 36:1-13. 2007.
8. Barua, H., P. K. Biswas, K. E. Olsen, S. K. Shil, and J. P. Christensen. Molecular characterization of motile serovars of Salmonella enterica from breeder and commercial broiler poultry farms in Bangladesh. PLOS One 8: e57811. 2013.
9. Castillo-Torres, K. Y., D. P. Arnold, E. S. McLamore Rapid isolation of Escherichia coli from water samples using magnetic microdiscs. Sensors and Actuators B: Chemical, 2019 291:58-66. DOI: 10.1016/j.snb.2019.04.043.
10. Castillo-Torres, K. Y., E. S. McLamore, and D. P. Arnold. A High-Throughput Microfluidic Magnetic Separation (microFMS) Platform for Water Quality Monitoring. Micromachines (Basel) 11. 2019.
11. CDC. Centers for Disease Control and Prevention. https://www.cdc.gov/salmonella/Braenderup-04-18/index.html.
12. CDC. Centers for Disease Control and Prevention. OutbreakNet-Foodborne Outbreak Online Database. https://wwwn.cdc.gov/norsdashboard/.
13. CDC. Centers for Disease Control and Prevention. Snapshots of Salmonella Serotypes https://www.cdc.gov/salmonella/reportspubs/salmonella-atlas/serotype-snapshots.html.
14. CDC. Centers for Diseases Control and Prevention, Healthy People 2020 Progress Report https://www.cdc.gov/narms/reports/healthy-people-2020.html. In.
15. CDC. Centers for Diseases Control and Prevention, An Atlas of Salmonella in the United States, 1968-2011 https://www.cdc.gov/salmonella/pdf/salmonella-atlas-508c.pdf. In.
16. CDC. Multistate Outbreak of Human Salmonella Enteritidis Infections Associated with Shell Eggs. http://www.cdc.gov/salmonella/enteritidis.
17. Chaudhari, A. A., C. V. Jawale, S. W. Kim, and J. H. Lee. Construction of a Salmonella gallinarum ghost as a novel inactivated vaccine candidate and its protective efficacy against fowl typhoid in chickens. Vet Res 43:44. 2012.¹
18. Chittick, P., A. Sulka, R. V. Tauxe, and A. M. Fry. A summary of national reports of foodborne outbreaks of Salmonella Heidelberg infections in the United States: clues for disease prevention. J Food Prot 69:1150-1153. 2006.
19. De Buck, J., F. Van Immerseel, F. Haesebrouck, and R. Ducatelle. Protection of laying hens against Salmonella Enteritidis by immunization with type 1 fimbriae. Vet Microbiol 105:93-101. 2005.
20. Denagamage, T. N., E. Wallner-Pendleton, B. M. Jayarao, L. Xiaoli, E. G. Dudley, D. Wolfgang, and S. Kariyawasam. Detection of CTX-M-1 extended-spectrum beta-lactamase among ceftiofur-resistant Salmonella enterica clinical isolates of poultry. J Vet Diagn Invest 31:681-687. 2019.
21. Desin, T. S., W. Koster, and A. A. Potter. Salmonella vaccines in poultry: past, present and future. Expert Rev Vaccines 12:87-96. 2013.
22. Desin, T. S., A. L. Wisner, P. K. Lam, E. Berberov, C. S. Mickael, A. A. Potter, and W. Koster. Evaluation of Salmonella enterica serovar Enteritidis pathogenicity island-1 proteins as vaccine candidates against S. Enteritidis challenge in chickens. Vet Microbiol 148:298-307. 2011.
23. FDA. Food and Drug Administration. 2010. National Antimicrobial Resistance Monitoring System-enteric bacteria (NARMS): 2007 executive report. U.S. Department of Health and Human Services, FDA, Rockville, MD.
24. FDA. U.S. Food and Drug Administration. Bacteriological Analytical Manual. Chapter 5: Salmonella. February 2011. http://www.fda.gov/Food/ScienceResearch/LaboratoryMethods/BacteriologicalAnalyticalManua IBAM.
25. FDA. U.S. Food and Drug Administration. https://www.fda.gov/food/laboratorymethods-food/testing-methodology-salmonella-enteritidis-se.
26. FSN. Food Safety News. https://www.foodsafetynews.com/2013/08/the-five-mostcommon-salmonella-strains.
27. Garland, N. T., E. S. McLamore, N. D. Cavallaro, D. Mendivelso-Perez, E. A. Smith, D. Jing, J. C. Claussen. Flexible Laser-Induced Graphene for Nitrogen Sensing in Soil. Advanced Functional Materials. 2018 10 (45): 39124-39133. DOI: 10.1021/acsami.8b10991.
28. Gast, R. K. Serotype-specific and serotype-independent strategies for preharvest control of food-borne Salmonella in poultry. Avian Dis 51:817-828. 2007.
29. Gingerich, E. Preventing Salmonella Enteritidis (SE) in egg layer flocks. http://www.diamondv.com/newscenter_documents/NutritionLineFeatureArticles/Poultry2010/July2010_Preventing % 20Salmonella%20Enteritidis%20 in %20Egg %20Layer %20Flocks_Gingerich. pdf.
30. Gingerich, E. Reduction of Salmonella Enteritidis in Commercial Layers, https://www.fsis.usda.gov/wps/wcm/connect/e2f8a230-44dc-41cb-b8e9-f99c8c4fe8ef/Gingerich_092908.pdf?MOD=AJPERES.
31. Gingerich, E. Salmonella in the egg laying industry. In: Salmonella in Poultry: Epidemiology, Regulations, Detection and Interventions. American College of Poultry Veterinarians and Western Poultry Disease Conference. 2009 Mar. 22. Davis CA.
32. Henzler, D. J., M. Henninger, and P. Debok. A five-year (1994-1999) critical analysis of the Pennsylvania egg quality assurance program (PEQAP). Proceedings of the 1999 American Veterinary Medical Association/American Association of Avian Pathologist Annual Meetings; 1999 Jul. 10; New Orleans, LA. JAVMA. 1999:215. Abstract #45.²
33. Hogue, A., P. White, J. Guard-Petter, W. Schlosser, R. Gast, E. Ebel, J. Farrar, T. Gomez, J. Madden, M. Madison, A. M. McNamara, R. Morales, D. Parham, P. Sparling, W. Sutherlin, and D. Swerdlow. Epidemiology and control of egg-associated Salmonella Enteritidis in the United States of America. Rev Sci Tech 16:542-553. 1997.
34. Hu, J., L. J. Worrall, M. Vuckovic, C. Hong, W. Deng, C. E. Atkinson, B. Brett Finlay, Z. Yu, and N. C. J. Strynadka. T3S injectisome needle complex structures in four distinct states reveal the basis of membrane coupling and assembly. Nat Microbiol 4:2010-2019. 2019.
35. Kaniga, K., J. C. Bossio, and J. E. Galan. The Salmonella Typhimurium invasion genes invF and invG encode homologues of the AraC and PulD family of proteins. Mol Microbiol 13:555-568. 1994.
36. Kariyawasam, S., B. N. Wilkie, and C. L. Gyles. Construction, characterization, and evaluation of the vaccine potential of three genetically defined mutants of avian pathogenic Escherichia coli. Avian Dis 48:287-299. 2004.
37. Kariyawasam, S., B. N. Wilkie, and C. L. Gyles. Resistance of broiler chickens to Escherichia coli respiratory tract infection induced by passively transferred egg-yolk antibodies. Vet Microbiol 98:273-284. 2004.
38. Kariyawasam, S., B. N. Wilkie, D. B. Hunter, and C. L. Gyles. Systemic and mucosal antibody responses to selected cell surface antigens of avian pathogenic Escherichia coli in experimentally infected chickens. Avian Dis 46:668-678. 2002.
39. Kradel, D., D. Henzler, and M. Hanninger. Salmonella Enteritidis (S. Enteritidis) experiences with a control program. Proceedings of the 47th North Central Avian Disease Conference and Symposium on Making and Evaluating Health Management Decisions; 1996 Sep. 29-Oct. 1; Columbus, OH.
40. Kradel, D. C. Salmonella Enteritidis control: egg industry perspective. Presented at the 46th Annual New England Poultry Health Conference; 1997 Mar. 26; Portsmouth, NH.
41. Mason, J. Salmonella Enteritidis control programs in the United States. Int J Food Microbiol 21:155-169. 1994.
42. Moreau, M. R., D. S. Wijetunge, M. L. Bailey, S. R. Gongati, L. L. Goodfield, E. M. Hewage, M. J. Kennett, C. Fedorchuk, Y. V. Ivanov, J. E. Linder, B. M. Jayarao, and S. Kariyawasam. Growth in egg yolk enhances Salmonella Enteritidis colonization and virulence in a mouse model of human colitis. PLOS One 11: e0150258. 2016.
43. Moreau, M. R., D. S. Wijetunge, E. M. Kurundu Hewage, B. M. Jayarao, and S. Kariyawasam. Genome Sequences of two strains of Salmonella enterica sSerovar Enteritidis isolated from shell eggs. Genome Announc 3. 2015.
44. Mumma, G. A., P. M. Griffin, M. I. Meltzer, C. R. Braden, and R. V. Tauxe. Egg quality assurance programs and egg-associated Salmonella Enteritidis infections, United States. Emerg Infect Dis 10:1782-1789. 2004.
45. Nidaullah, H., N. Abirami, A. K. Shamila-Syuhada, L. O. Chuah, H. Nurul, T. P. Tan, F. W. Z. Abidin, and G. Rusul. Prevalence of Salmonella in poultry processing environments in wet markets in Penang and Perlis, Malaysia. Vet World 10:286-292. 2017.
46. ODPHP. Office of Disease Prevention and Health Promotion, Healthy People. https://www.healthypeople.gov/2020/topics-objectives/topic/food-safety/objectives.
47. Okamura, M., W. Matsumoto, F. Seike, Y. Tanaka, C. Teratani, M. Tozuka, T. Kashimoto, K. Takehara, M. Nakamura, and Y. Yoshikawa. Efficacy of soluble recombinant FliC protein from Salmonella enterica serovar Enteritidis as a potential vaccine candidate against homologous challenge in chickens. Avian Dis 56:354-358. 2012.³
48. Papezova, K., H. Havlickova, F. Sisak, v. Kummer, M. Faldyna, and i. Rychlik. Comparison of live and inactivated Salmonella Typhimurium vaccines containing combinations of SPi-1 and SPi-2 antigens in poultry. Vet. Med. Prague 2008 53:315-323.
49. Renwick, S. A., R. J. Irwin, R. C. Clarke, W. B. McNab, C. Poppe, and S. A. McEwen. Epidemiological associations between characteristics of registered broiler chicken flocks in Canada and the Salmonella culture status of floor litter and drinking water. Can Vet J 33:449-458. 1992.
50. Scallan, E., R. M. Hoekstra, F. J. Angulo, R. V. Tauxe, M. A. Widdowson, S. L. Roy, J. L. Jones, and P. M. Griffin. Foodborne illness acquired in the United States—major pathogens. Emerg Infect Dis 17:7-15. 2011.
51. Schlosser, W. D., D. J. Henzler, J. Mason, D. Kradel, L. Shipman, S. Trock, S. H. Hurd, A. T. Hogue, W. Sischo, and E. D. Ebel. The Salmonella enterica serovar Enteritidis pilot project. In: Salmonella enterica serovar Enteritidis in humans and animals: epidemiology, pathogenesis, and control. A. M. Saeed, R. M. Gast, M. R. Potter, P. J. Wall, eds. Iowa State University Press, Ames, IA. pp. 353-365. 1999.
52. Shivaprasad, H. L., J. F. Timoney, S. Morales, B. Lucio, and R. C. Baker. Pathogenesis of Salmonella Enteritidis infection in laying chickens. I. Studies on egg transmission, clinical signs, fecal shedding, and serologic responses. Avian Dis 34:548-557. 1990.
53. Snoeyenbos, G. H., C. F. Smyser, and H. Van Roekel. Salmonella infections of the ovary and peritoneum of chickens. Avian Dis 13:668-670. 1969.
54. St Louis, M. E., D. L. Morse, M. E. Potter, T. M. DeMelfi, J. J. Guzewich, R. V. Tauxe, and P. A. Blake. The emergence of grade A eggs as a major source of Salmonella Enteritidis infections. New implications for the control of salmonellosis. JAMA 259:2103-2107. 1988.
55. Tan, S., C. L. Gyles, and B. N. Wilkie. Evaluation of an aroA mutant Salmonella Typhimurium vaccine in chickens using modified semisolid Rappaport Vassiliadis medium to monitor faecal shedding. Vet Microbiol 54:247-254. 1997.
56. Tauxe, R. V., M. P. Doyle, T. Kuchenmuller, J. Schlundt, and C. E. Stein. Evolving public health approaches to the global challenge of foodborne infections. Int J Food Microbiol 139 Suppl 1: S16-28. 2010.
57. Thiagarajan, D., A. M. Saeed, and E. K. Asem. Mechanism of transovarian transmission of Salmonella Enteritidis in laying hens. Poult Sci 73:89-98. 1994.
58. Timoney, J. F., H. L. Shivaprasad, R. C. Baker, and B. Rowe. Egg transmission after infection of hens with Salmonella Enteritidis phage type 4. Vet Rec 125:600-601. 1989.
59. Toyota-Hanatani, Y., Y. Kyoumoto, E. Baba, T. Ekawa, H. Ohta, H. Tani, and K. Sasai. Importance of subunit vaccine antigen of major Fli C antigenic site of Salmonella Enteritidis II: a challenge trial. Vaccine 27:1680-1684. 2009.
60. Trepka, M. J., J. R. Archer, S. F. Altekruse, M. E. Proctor, and J. P. Davis. An increase in sporadic and outbreak-associated Salmonella Enteritidis infections in Wisconsin: the role of eggs. J Infect Dis 180:1214-1219. 1999.
61. UEP. United Egg Producers. UEP “5-STAR” total quality assurance program: a HACCP type food safety program with validation. Atlanta: United Egg Producers; 2001.
62. USDA. Food Safety Inspection Service. Salmonella Enteritidis risk assessment: shell eggs and egg products. Washington: U.S. Department of Agriculture; 1998.
63. USDA. The FSIS Salmonella Action Plan: A Two Year Update 2016 https://www.fsis.usda.gov/wps/portal/fsis/topics/food-safety-education/get-answers/food-safetyfact-sheets/foodborne-illness-and-disease/salmonella/sap-two-year.⁴
64. USDA. U.S. Department of Agriculture. 1997. The National poultry improvement plan and auxiliary provisions. U.S. Department of Agriculture, Animal and Plant Health Inspection Service, Washington, DC.
65. USDA. U.S. Department of Agriculture/Animal and Plant Inspection Service. Salmonella enterica serotype Enteritidis in table egg layers in the U.S. National Animal Health Monitoring System (NAHMS). Conyers (GA): the Department; 2000.
66. USDA. United States Department of Agriculture Food Safety and Inspection Service https://www.fsis.usda.gov/wps/portal/fsis/topics/food-safety-education/get-answers/food-safetyfact-sheets/foodborne-illness-and-disease/salmonella.
67. USDA. United States Department of Agriculture. National Poultry Improvement Plan program Standards http://www.poultryimprovement.org/documents/ProgramStandardsA-E.pdf.
68. Vaezirad, M. M., M. G. Koene, J. A. Wagenaar, and J. P. M. van Putten. Chicken immune response following in ovo delivery of bacterial flagellin. Vaccine 36:2139-2146. 2018.
69. Vandeputte, J., A. Martel, N. Van Rysselberghe, G. Antonissen, M. Verlinden, L. De Zutter, M. Heyndrickx, F. Haesebrouck, F. Pasmans, and A. Garmyn. In ovo vaccination of broilers against Campylobacter jejuni using a bacterin and subunit vaccine. Poult Sci 98:5999-6004. 2019.
70. Vanegas, D. C., L. Patiño, C. Mendez, D. Alves de Oliveira, A. M. Torres, E. S. McLamore, C. Gomes. Low-cost electrochemical biosensor for detection of biogenic amines in food samples. Biosensors Journal, 8 (2). DOI: 10.3390/bios8020042. 2018.
71. Voetsch, A. C., T. J. Van Gilder, F. J. Angulo, M. M. Farley, S. Shallow, R. Marcus, P. R. Cieslak, V. C. Deneen, and R. V. Tauxe. FoodNet estimate of the burden of illness caused by nontyphoidal Salmonella infections in the United States. Clin Infect Dis 38 Suppl 3: S127-134. 2004.
72. WHO. World Health Organization & Food and Agriculture Organization of the United Nations. 2016. Interventions for the control of non-typhoidal Salmonella spp. in beef and pork: meeting report and systematic review. World Health Organization, Geneva, Switzerland. http://www.who.int/iris/handle/10665/249529.
73. WHO. World Health Organization, Foodborne Disease Burden Epidemiology Reference Group. 2015. WHO estimates of the global burden of foodborne diseases. World Health Organization, Geneva, Switzerland.
74. Wisner, A. L., T. S. Desin, P. K. Lam, E. Berberov, C. S. Mickael, H. G. Townsend, A. A. Potter, and W. Koster. Immunization of chickens with Salmonella enterica subspecies enterica serovar Enteritidis pathogenicity island-2 proteins. Vet Microbiol 153:274-284. 2011.
75. Zhang, G., E. Thau, E. W. Brown, and T. S. Hammack. Comparison of a novel strategy for the detection and isolation of Salmonella in shell eggs with the Food and Drug Administration Bacteriological Analytical Manual method. Poult Sci 92:3266-3274. 2013.

What is claimed is:

1. An immunogenic composition that comprises: an immunogenically effective amount of a Salmonella Enteritidis protein InvG, and optionally a pharmaceutically acceptable carrier.

2. The composition according to claim 1 wherein said Salmonella Enteritidis protein InvG comprises a polypeptide sequence comprising SEQ ID NO. 2 or immunogenic fragments or variants thereof.

3. A composition for inducing an immune response against Salmonella Enteritidis comprising an organism engineered to express Salmonella Enteritidis protein InvG, and optionally a pharmaceutically acceptable carrier.

4. The composition of claim 3, wherein the organism populates in a poultry subject.

5. The composition of claim 3, where the organism is E. coli.

6. The composition of claim 5, where the E. coli comprises an avirulent mutation.

7. The composition of claim 6, where the avirulent mutation comprises SEQ ID NO:3.

8. The composition according to claim 3 wherein said Salmonella Enteritidis protein InvG is cloned into an expression vector that is engineered to express SEQ ID NO. 2 or immunogenic fragments or variants thereof.

9. A method for inducing an immune response in a poultry subject against a disease or infection caused by S. enterica, the method comprising administering to said poultry subject an immunogenically effective dose of the compositions according to any of claims 1-8.

10. The method of claim 9, wherein administering the composition according to claims 3-8 further comprises an immunogenic response against E. coli.

11. The method of claim 9, wherein the poultry subject is a subject selected from the group consisting of chickens; ducks; geese; turkeys; bantams; quail; pheasant; and pigeons

12. The method of claim 9 wherein the composition is administered orally, intramuscularly, or in ovo.

13. The method of claim 9 wherein the composition of claims 1-2 is administered with an adjuvant.

14. The method of claim 9 wherein the composition is administered in multiple dose 14 days apart.

15. The method of claim 9 wherein administering comprises an immunogenic response against S. Enteritidis, S. Typhimurium, S. Heidelberg, or S. Braenderup.

16. A method for determining the presence of Salmonella comprising: filtering a sample through an electrically active carbon filter biofunctionalized with capture probes targeting InvG-T3SS.

17. The method of claim 16 wherein the filter is comprised of emulsion-coated cellulose paper or nitrocellulose membranes.

18. The method of claim 16 wherein the filter is perforated with microholes.

19. The method of claim 18 wherein the perforation is performed with a Nd:YAG laser.

20. The method of claim 18 wherein the microholes are metallized with platinum or gold.

21. The method of claim 16 wherein the InvG-T3SS capture probes are comprised of antibodies, aptamers, lectins, or oligopeptides.

22. The method of claim 16 wherein the sample is filtered using vacuum or gravity filtration.

23. The method of claim 16 wherein the sample comprises a viscous fluid including, but not limited, to egg yolk, creams, broths, or stocks.

24. The method of claim 16 wherein the presence of Salmonella is verified using standard electrochemical impedance spectroscopy, PCR, cell culture, or other Association of Official Agricultural Chemists (AOAC) methods known in the arts.

25. The composition of any of claims 3-8, wherein the avirulent aroA strain is APEC strain PSUO78 comprising an aroA and/or asd mutation.

26. The composition of claim 25, wherein the aroA and/or asd mutation comprises a deletion of aroA and asd genes.

27. The composition of claim 25 or claim 26, wherein the expression vector is pBR322 into which SEQ ID NO: 2 is inserted.

28. The composition of any of claims 3-8 and 27, wherein expression of SEQ ID NO: 2 is driven by a promoter in the expression vector.

29. The composition of any of claims 1-8, further comprising an adjuvant.


SEQUENCE LISTING (10 sequences)

SEQ ID NO: 1 - DNA, 1689 bp, Salmonella enterica
  source 1..1689: mol_type = other DNA
agtaaattaacggaggactggagataggtctatttgtgcttgggtaaagacattgaatagcaggggtgaggtccgtggcgaaacgaagtcttataacaaatggctaagcgaccgtaggccgcaattgcccagctgttaaagaaaaccaagttagtacttgtgtgcttgttgtaatgaaaataagaatgaccttatcgccttgtccgatggttattcgccattaaacggatttttgccttatgaaacctgtcatagccataaacgtagggcacatattggtggctggtcgtttgaaaaaggcacgccgtgagagcgttatcacgattaattgcaagcgggctgaagcccattgcgtagatgcctccaccatcatagcctaacgccgcagaatagcaacggtagaagttacaggtcgctgtaaagatagactggtagacgcctttttgccccgtcgtgagcctagtaacaaggcatacaatgtacgagttcgcggtgtaatgcaaggggttagtcgaaccacattttgcaagacaacaatagtttttatcgcccttgtaaaaggacccagtcattatggcccgcactttggtggcagcggacaaagaagagaagattgcgtaactggcgccgctacttagctgacggtagctcccatgaataacttctgaccaaattactgtgcggttcaaacaggggttatcattacgacggcgaggtacttcacgggtttgcgaggtctagcgaaaataattctagctgttaggtgtccctattaagatgcactgcaaaccgctgtaggtcgcgaaattggtcgtaaagctattttacgtggacgagtcggcaagggaaatgattgtttgacaaccataggcctatccggtgctaaaattataacgggcggcgccgtaaaacgaaatttcgaaggacgtctgagtacggtggccgtattaaacgacgaaatggaaaaagaggtaagcgacttttgcgaccgtagcggcccccaagcgatgactgttataatggatttccaacgagaagagggacgttgttggaaagttaccggcaccgttatggcccctattggtaaaagactagcgcgtctaatatccatgctagcgggtgcttccataacaagtctgcgtagtggggataaaagactgcagggtcgagttatggtagcaaaacgaacaggtagtaccaccgccgcaactggtggtatagttgtatctgcccaccgggactttgtatcttacaaggaaatgccaatagcggtgcatcgcccattaaaaacaatatatttggactcgcaaaatcttttaacaacttgagtaactcactctgcaacgcatttctttggtgccgtaacgcgtaaagtgaccgcagtatttatatctatcggacgggtagctttatggtttagtcggggtcaacatccctttcgaagaggtcattacgcaatcctagcactttgagtttcaacgggcattaaaaaaaagcacggcggtaaaacgattgttactgtccgaggaaatcgacatcgcggtaccgtagctttttacaggcgtccgatagcagaaagcgttgtttgggtgaagggcaatgtccataaaaaagtgatcttattggtccacattggtcttgttcgcgccgtgtacggtcgtgagaccggttttcttatacacagaagta

SEQ ID NO: 2 - protein, 562 aa, Salmonella enterica
MKTHILLARVLACAALVLVTPGYSSEKIPVTGSGFVAKDDSLRTFFDAMALQLKEPVIVSKMAARKKITGNFEFHDPNALLEKLSLQLGLIWYFDGQAIYIYDASEMRNAVVSLRNVSLNEFNNFLKRSGLYNKNYPLRGDNRKGTFYVSGPPVYVDMVVNAATMMDKQNDGIELGRQKIGVMRLNNTFVGDRTYNLRDQKMVIPGIATAIERLLQGEEQPLGNIVSSEPPAMPAFSANGEKGKAANYAGGMSLQEALKQNAAAGNIKIVAYPDTNSLLVKGTAEQVHFIEMLVKALDVAKRHVELSLWIVDLNKSDLERLGTSWSGSITIGDKLGVSLNQSSISTLDGSRFIAAVNALEEKKQATVVSRPVLLTQENVPAIFDNNRTFYTKLIGERNVALEHVTYGTMIRVLPRFSADGQIEMSLDIEDGNDKTPQSDTTTSVDALPEVGRTLISTIARVPHGKSLLVGGYTRDANTDTVQSIPFLGKLPLIGSLFRYSSKNKSNVVRVFMIEPKEIVDPLTPDASESVNNILKQSGAWSGDDKLQKWVRVYLDRGQEAIK

SEQ ID NO: 3 - DNA, 592 bp, Salmonella enterica
  source 1..592: mol_type = other DNA
atggaatccctgacgttacaacccatcgctcgtgtcgatggcactattaatctgcccggttccaagagcgtttctaaccgcgctttattgctggcggcattagcacacggcaaaacagtattaaccaatctgctggatagcgatgacgtgcgccatatgctgaatgcattaacagcgttaggggtaagctatacgctttcagccgatcgtacgcgttgcgaaattatcggtaacggcggtccattacacgcagaaggtgattgccacggcggcgttatttgcaaaaggcaccaccacgctgcgcaatatctataactggcgtgttaaagagaccgatcgcctgtttgcgatggcaacagaactgcgtaaagtcggcgcggaagtggaagaggggcacgattacattcgtatcactcctccggaaaaactgaactttgccgagatcgcgacatacaatgatcaccggatggcgatgtgtttctcgctggtggcgttgtcagatacaccagtgacgattcttgatcccaaatgcacggccaaaacatttccggattatttcgagcagctggcgcggattagccaggcagcctga

SEQ ID NO: 4 - DNA, 4361 bp, synthetic construct
  misc_feature 1..4361: note = plasmid pBR322
  source 1..4361: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 5 - DNA, 29 bp, synthetic construct
  misc_feature 1..29: note = galE primer
  source 1..29: mol_type = other DNA
ccatggtaccgttacgcgccagttcagtt

SEQ ID NO: 6 - DNA, 30 bp, synthetic construct
  misc_feature 1..30: note = galE primer
  source 1..30: mol_type = other DNA
ggccgagctcttggaagtcatacctgtgtg

SEQ ID NO: 7 - DNA, 30 bp, synthetic construct
  misc_feature 1..30: note = purA primer
  source 1..30: mol_type = other DNA
gcgcggtacctactctcgtaatcaacggtg

SEQ ID NO: 8 - DNA, 30 bp, synthetic construct
  misc_feature 1..30: note = purA primer
  source 1..30: mol_type = other DNA
ggccgagctcagtgaagaccagttgaccat

SEQ ID NO: 9 - DNA, 30 bp, synthetic construct
  misc_feature 1..30: note = aroA primer
  source 1..30: mol_type = other DNA
ccatggtacctcgtgtcgatggcactatta

SEQ ID NO: 10 - DNA, 30 bp, synthetic construct
  misc_feature 1..30: note = aroA primer
  source 1..30: mol_type = other DNA
ggccgagctctcaagaatcgtcactggtgt